(19) United States
(12) Patent Application Publication
(10) Pub. No.: US 2022/0369736 A1
(43) Pub. Date: Nov. 24, 2022

(54) PROTECTIVE EYEWEAR AND MEDICAL FACE MASK SYSTEM, AND METHOD FOR PROTECTING THE EYES AND STABILIZING A FACE MASK DURING A MEDICAL PROCEDURE

(71) Applicant: Edward Lehman, Las Vegas, NV (US)

(72) Inventor: Edward Lehman, Las Vegas, NV (US)

(21) Appl. No.: 17/679,810

(22) Filed: Feb. 24, 2022

Related U.S. Application Data
(60) Provisional application No. 63/154,302, filed on Feb. 26, 2021.

(51) Int. Cl. A41D 13/11; A41D 31/18

(52) U.S. Cl. CPC A41D 13/1184; A41D 13/1161; A41D 31/18

(57) ABSTRACT

A pair of protective eyewear for securing a medical face mask on a face of a patient to prevent migration of the medical face mask about the face of the patient during a medical or surgical procedure. The protective eyewear may include a main body having a nosepiece and a pair of eye portions secured in the main body. The nosepiece may include a fastener defining a mask-receiving space for releasable receipt of a ridge of a medical face mask. The eyewear may additionally include one or more straps or other head securing structure, extending from the main body, to secure the eyewear to a head of the patient. Further, there is provided a system protecting eyes of a patient during a medical or surgical procedure and a method of securing a face mask to a face of a patient to prevent migration of the medical face mask about the face of the patient during the medical or surgical procedure.

### CROSS-REFERENCE TO RELATED APPLICATION

This application claims the benefit of U.S. Provisional Patent Application No. 63/154,302, filed on Feb. 26, 2021, which is incorporated herein by reference in its entirety.

### FIELD OF THE INVENTION

The present invention relates generally to protective eyewear and medical face masks for use during medical and surgical procedures, and more particularly, to protective eyewear releasably engageable with therapeutic, diagnostic or other medical face masks to prevent movement or migration of the medical face mask about a face of a patient during a medical or surgical procedure.

### BACKGROUND OF THE INVENTION

During medical procedures, it is often necessary to use a medical face mask to cover and protect the mouth and nose area of a patient and to administer oxygen and/or anesthesia to the patient. These medical face masks are often secured to the head of the patient by one or more straps to prevent movement or “migration” of the mask along and relative to the face of the patient during the procedure.

Often, the medical face mask is connected to a variety of external scopes and/or tubes to receive the anesthesia as well as ventilation, hydration, suction, etc. Often, these scopes and tubes pull relatively strongly on the medical face mask during the procedure, resulting in the medical face mask moving or “migrating” about the face of the patient during the procedure.

Additionally, it has been widely observed that patients tend to rub their eyes instinctively or even automatically during the procedure, and most especially, while recovering from anesthesia. On many occasions, the rubbing is so intense that leads to ocular abrasion, which in turn causes significant patient discomfort during recovery from the medical procedure or surgery. In fact, ocular abrasion is a common side effect of procedures involving anesthesia or sedation. Various types of goggles or other eyewear are often provided to cover the eyes of the patient to prevent harm to the eyes during the procedure. However, these goggles or eyewear may interfere with the placement of the medical face mask about the face of the patient causing the goggles or eyewear to move the medical face mask about the face of the patient. I.e., conventional goggles may worsen medical face mask migration.

Accordingly, there is need for a solution to at least one of the aforementioned problems. For instance, there is an established need for a medical device to prevent migration of a medical face mask about a face of a patient during a medical or surgical procedure. There is still further an established need for eyewear to protect eyes of a patient during a medical or surgical procedure without disrupting the positioning of the medical face mask on the face of the patient.

### SUMMARY OF THE INVENTION

The present invention is directed to protective eyewear and a system for securing a therapeutic, diagnostic or other medical face mask (hereinafter referred to generically as ‘medical face mask’) on a face of a subject to prevent migration of the medical face mask about the face of the subject during a medical or surgical procedure. The protective eyewear includes a main body having a centralized connecting point or nosepiece and a pair of eye portions secured in the main body. The eye portions may be flat or curved panels; the panels may be transparent, translucent, opaque, or combinations thereof. The centralized connecting point or nosepiece includes a fastener defining one or more mask-receiving spaces for releasable receipt of a portion of a medical face mask. The eyewear may additionally include one or more straps, bands, or harnesses extending from the main body to secure the eyewear to the subject's head. There is also disclosed a system and method of securing a face mask to a face of a subject to prevent migration of the medical face mask about the face of the subject during the medical or surgical procedure.

In a first implementation, a protective eyewear for stabilizing a medical face mask on a face of a subject may include a main body, a head securing structure, and a fastener. The main body may be configured to fit on a subject's face over at least one eye of the subject. The head securing structure may be carried by the main body, and may be configured to secure the main body to the subject's head. The fastener may be carried by the main body, and may define one or more mask-receiving spaces for releasably receiving and securing of a portion of a medical face mask.

In a second aspect, the main body may include at least one eye portion configured to extend over and cover one or both eyes of the subject.

In another aspect, the main body may be configured to enclose both eyes of the subject.

In another aspect, the head securing structure may be elastic.

In another aspect, the head securing structure may be configured to wrap around the subject's head.

In yet another aspect, the head securing structure may include an upper elongated element and a lower elongated element defining a respective upper loop and lower loop. The upper and lower loops may each extend from the main body and may each be configured to wrap around the subject's head.

In another aspect, the main body may include a nosepiece configured to extend over the subject's nose. The fastener may be provided at the nosepiece.

In another aspect, the one or more mask-receiving spaces may include an elongated slot arcuately formed along the nosepiece.

In another aspect, the one or more mask-receiving spaces may be configured to releasably receive a ridge of the medical face mask.

In yet another aspect, the one or more mask-receiving spaces may be defined by a first portion and a second portion of the fastener. The first portion may extend frontward from the main body. The second portion may extend downward and rearward from the first portion and at an angle with the first portion.

In another aspect, the first portion may extend downward from the main body.

In another implementation of the invention, a system for protecting the eyes of a subject and providing a stabilized medical face mask on the face of the subject may include a medical face mask and a protective eyewear. The medical face mask may include a mask body configured to fit over the nose and mouth of a subject, and a strap extending from the mask body and configured to secure the mask body to the subject's head. The protective eyewear may include a main body configured to fit on the subject's face over at least one eye of the subject, and a head securing structure carried by the main body, the head securing structure configured to secure the main body to the subject's head. The protective eyewear may further include a fastener carried by the main body, the fastener configured to attach to the mask body of the medical face mask.

In another aspect, the main body of the protective eyewear may include a nosepiece configured to extend over the subject's nose. The fastener may be provided at the nosepiece and configured to releasably receive and secure a top portion of the mask body of the medical face mask.

In another aspect, the fastener may include an elongated slot arcuately formed along the nosepiece. The elongated slot may be configured to releasably receive and secure an arcuate top portion of the mask body of the medical face mask.

In yet another implementation of the invention, a method of stabilizing a medical face mask on a face of a subject may include the steps of: positioning a medical face mask on a subject's head, such that a mask body of the medical face mask may be positioned over the nose and mouth of the subject; positioning a protective eyewear on the subject's head, such that a main body of the protective eyewear covers at least one eye of the subject and a head securing structure of the protective eyewear secures the main body to the subject's head; and preventing or reducing migration of the mask body of the medical face mask along the face of the subject by securing the protective eyewear to the mask body.

In another aspect, the step of preventing or reducing migration of the mask body may include securing the main body of the protective eyewear to the mask body of the medical face mask.

In another aspect, the protective eyewear may include a fastener configured to releasably attach to the mask body of the medical face mask. The step of preventing or reducing migration of the mask body may include securing the fastener to the mask body.

In another aspect, the fastener may include one or more mask-receiving spaces. The step of preventing or reducing migration of the mask body may include releasably receiving and securing a portion of the mask body within the one or more mask-receiving spaces.

In another aspect, the step of positioning the protective eyewear may include securing a head securing structure of the protective eyewear to the subject's head. The step of preventing or reducing migration of the mask body may include overlapping the head securing structure of the protective eyewear over a strap of the medical face mask.

In yet another aspect, the step of positioning the medical face mask may include securing the strap of the medical face mask to the head of the subject.

These and other objects, features, and advantages of the present invention will become more readily apparent from the attached drawings and the detailed description of the preferred embodiments, which follow.

### BRIEF DESCRIPTION OF THE DRAWINGS

The preferred embodiments of the invention will hereinafter be described in conjunction with the appended drawings provided to illustrate and not to limit the invention, where like designations denote like elements, and in which:

FIG. 1 presents a top, front isometric view of protective eyewear for use with a medical face mask in accordance with an illustrative embodiment of the present invention;

FIG. 2 presents a bottom, rear isometric view of the protective eyewear of FIG. 1;

FIG. 3 presents a top, front isometric view of the protective eyewear of FIG. 1 prior to attachment with the medical face mask;

FIG. 4 presents a cross-sectional side elevation view of the assembled protective eyewear and medical face mask of FIG. 1;

FIG. 5 presents a side elevation view of the protective eyewear and medical face mask of FIG. 1 positioned on the head of a subject;

FIG. 6 presents a front elevation view of the protective eyewear and surgical mask of FIG. 1 positioned on the head of the subject; and

FIG. 7 presents a top, front isometric view of the protective eyewear of FIG. 1 in use with an alternative embodiment of a medical face mask.

Like reference numerals refer to like parts throughout the several views of the drawings.

### DETAILED DESCRIPTION

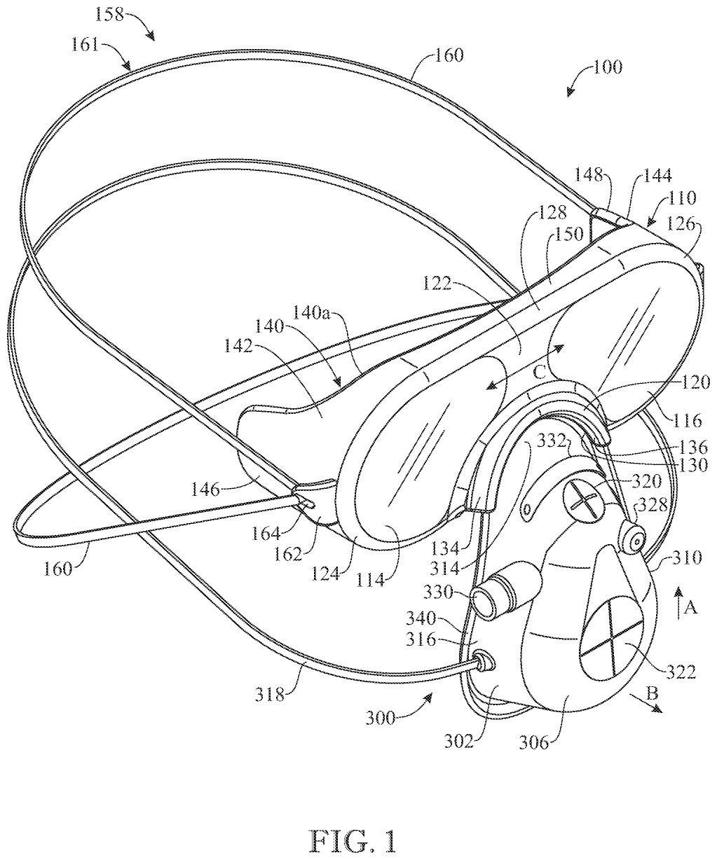

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. For purposes of description herein, the terms “upper”, “lower”, “left”, “rear”, “right”, “front”, “vertical”, “horizontal”, and derivatives thereof shall relate to the invention as oriented in FIG. 1. Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

The present invention is directed toward protective eyewear that can be releasably attached to a therapeutic, diagnostic, or other medical face mask (hereinafter referred to generically as ‘medical face mask’) to stabilize the medical face mask on the face of a patient during a medical procedure such as, but not limited to, a surgery. Shown throughout the figures are one or more illustrative embodiments of the invention.

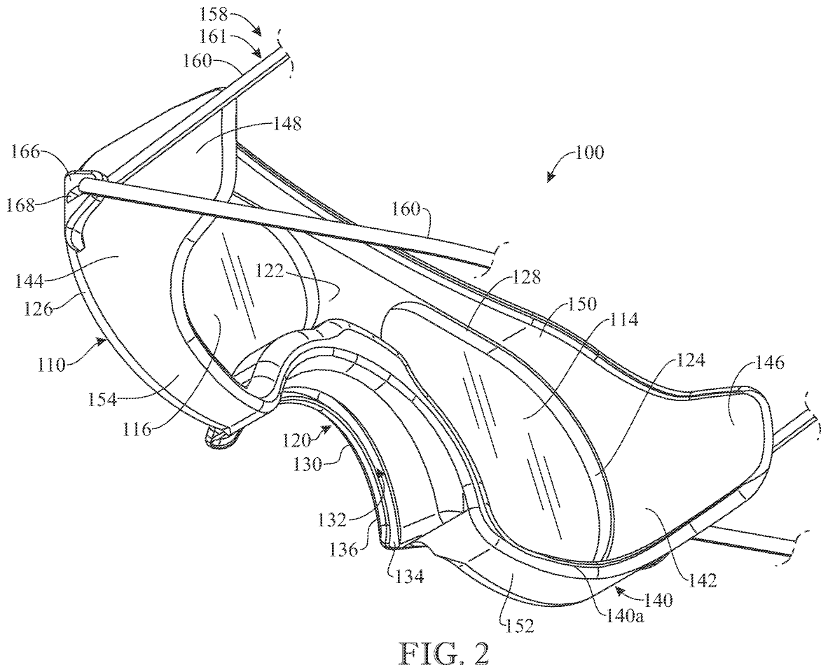

Referring to FIGS. 1-6, and initially to FIGS. 1 and 2, a pair of protective eyewear, hereinafter protective eyewear 100, is illustrated in accordance with a first exemplary embodiment of the present invention for use with a medical face mask, such as, but not limited to, a medical face mask 300 as shown. The protective eyewear 100 of the present disclosure releasably engages the medical face mask 300 to stabilize the medical face mask 300 on a face of a subject and reduce or prevent “migration” of the medical face mask 300, as described in more detail hereinbelow.

The protective eyewear 100 generally includes a frame or main body 110. In some embodiments, the main body 110 may have a first eye portion 114 and a second eye portion 116 configured to extend over and face the right and left eyes of a wearer of the protective eyewear 100, respectively. The first and second eye portions 114 and 116 may be flat, curved, or present alternative shapes, or combinations thereof. In different embodiments of the invention, the first and/or second eye portions 114, 116 may be transparent, translucent, tinted, colored, opaque, occlusive, or combinations thereof. In some embodiments, the first and/or second eye portions 114, 116 may be polarized, reflective, and/or radiography and/or laser protective. In further embodiments, the first and second eye portions may be fenestrated, slated or vented. The first and second eye portions 114 and 116, respectively, may be formed in a variety of shapes including circular, oval, etc. In some embodiments, the protective eyewear 100 may not include one or both of the first eye portion 114 and second eye portion 116; i.e., the protective eyewear may be devoid of an eye lens, wall, or covering extending over the eye. For instance, an opening may be provided and configure to register with the eye. In even further embodiments, the protective eyewear 100 may include a single large eye portion configured to extend over both eyes, the single large eye portion comprising both the first and second eye portions 114 and 116. For example, in some embodiments, the single large eye portion may cover both eyes and further extend over the face.

The main body 110 of the protective eyewear 100 of the present embodiment further includes a nosepiece 120, or portion or edge configured to be arranged on or proximate to the wearer's nose, and generally over and around an upper area of the nose and between supraorbital ridgeline or glabella (e.g., the bridge of the nose and between the eyebrows). A central connecting portion 122 may extend between, and interconnect, the first and second eye portions 114 and 116. The nosepiece 120 is arranged in a central area of the main body 110, and may extend, for instance, from and below the central connecting portion 122 and, optionally, from and generally between the first and second eye portions 114 and 116, as shown. In the disclosed embodiment, the first and second eye portions 114 and 116 are kidney-shaped and comprise a respective concavely curved edge generally conforming to and extending around the nosepiece 120.

With continued reference to FIG. 1, the main body 110 of the protective eyewear 100 may further include first and second side sections 124 and 126 arranged at an outer side or end of the first and second eye portions 114 and 116, respectively, and a top section 128 extending between the first and second side sections 124 and 126. For instance and without limitation, the first, second and top sections 124, 126 and 128 may provide a rearward curvature or curved edge to the main body 110. The first and second side sections 124 and 126 may extend from the top section 128, around the first and second eye portions 114 and 116, and along a bottom side of the first and second eye portions 114 and 116.

The nosepiece 120 of the present embodiment may extend between the first and second side sections 124 and 126 at said bottom side of the first and second eye portions 114 and 116. The nosepiece 120 comprises at least one fastener configured to releasably connect to the medical face mask 300. For instance, the nosepiece 120 of the present embodiment includes a single, relatively elongated fastener 130 which is releasably engageable with the medical face mask 300, as will be described in greater detail hereinafter.

In some embodiments, the first eye portion 114 and a second eye portion 116 of the main body 110 may be integrally formed with adjacent parts or the remainder of the main body 110, such as by single-material or bi-material plastics injection molding. Alternatively, the first and second eye portions 114 and 116 may be secured within the main body 110 and to adjacent parts of the main body 110. In a non-limiting example, the first and second eye portions 114 and 116, respectively, may be secured within the main body 110 by engagement with the nosepiece 120 and the top section 128. Specifically, the first eye portion 114 may be secured within the main body 110 by engagement with the nosepiece 120, the first side section 124 and the top section 128. Likewise, the second eye portion 116 may be secured within the main body 110 by engagement with the nosepiece 120, the second side section 126 and the top section 128. The engagement may be achieved by an adhesive, a mechanical connection (e.g. a male-female fitting), a snap fitting, a friction fitting, or combinations thereof, for instance and without limitation. Alternatively or additionally, the first and second eye portions 114 and 116 may be secured to the central connecting portion 122, such as by an adhesive, a mechanical connection (e.g. a male-female fitting), a snap fitting, a friction fitting, or combinations thereof.

As further shown in FIGS. 1 and 2, the main body 110 may include a perimetral skirt-like formation, or skirt 140, extending generally rearward relative to the first and second eye portions 114 and 116 and the central connecting portion 122, to protect the eyes from the top, bottom, and sides thereof and further prevent access by the wearer's fingers. The skirt 140 may include a first lateral shield 142 extending from the first side section 124 of the main body 110 and a second lateral shield 144 extending from the second side section 126 of the main body 110. The first and second lateral shields 142 and 144 are arranged at an angle with the first and second eye portions 114 and 116, such that the first and second lateral shields 142 and 144 protect the eyes generally from the sides, while the first and second eye portions 114 and 116 protect the eyes generally from the front. The first and second lateral shields 142 and 144 may include a first lateral shield flange 146 and a second lateral shield flange 148, respectively, which may protrude generally rearward along the area of the cheekbone (zygomatic bone) extending rearward from the eyes. The skirt 140 may further include a top shield 150 (FIG. 1) extending generally rearward from the top section 128, and first and second bottom shields 152 and 154 extending generally rearward from a bottom side of the first and second eye portions 114 and 116, respectively. The top shield 150, first and second lateral shields 142 and 144, first and second bottom shields 152 and 154, and the nosepiece 120 may be shaped and sized such that a rear edge 140a of the skirt 140 formed by rear edges of the top shield 150, first and second lateral shields 142 and 144, first and second bottom shields 152 and 154, and the nosepiece 120, is ergonomically shaped to conform to contour or surface of the wearer's head and form a seal around the eyes and nose of the wearer to prevent harm to the eyes. In some embodiments, the skirt 140 may be slated, perforated, fenestrated, or vented.

The protective eyewear 100 may be provided to a patient during surgery to protect the eyes of the patient from harm while optionally allowing the patient to see. In order to secure the main body 110 relative to the face of the patient, the protective eyewear 100 of the present embodiment further includes a head securing structure 158 configured to secure the main body 110 to the wearer's head. For instance, in some embodiments, such as the present embodiment, the head securing structure 158 may include at least one elongated element, which may be formed into at least one loop. For instance, the head securing structure 158 of the present embodiment includes at least one strap loop or strap 160. Alternative embodiments are contemplated, however, without departing from the scope of the present disclosure. For example, the head securing structure 158 configured to secure the main body 110 to the wearer's head may include other elongated elements such as, but not limited to, a band, a cord, a harness, or the like.

The head securing structure 158 (e.g., the at least one strap 160) may extend from the main body 110. In some embodiments, the at least one strap 160 may be concentric in nature. In some embodiments, the head securing structure 158 may be at least partially elastic and provide some compression against the head of the patient and thereby provide a relatively tight attachment to further stabilize the main body 110 on the head; for instance, the at least one strap 160 may be preferably elastic. In different embodiments, the head securing structure 158 may be secured to different areas of the main body 110, and in varying ways. For instance, in some embodiments, the main body 110 may include a first mount 162 and a second mount 166, to which the head securing structure 158 (e.g., the one or more straps 160) are attached. In a non-limiting example, the main body 110 of the figures features the first and second mounts 162 and 166 extending from the first and second lateral shields 142 and 144, respectively. Furthermore, as shown, the head securing structure 158 of the depicted embodiment specifically includes two strap loops or straps 160, which are formed by a single strap body 161. The single strap body 161 extends through a slot 164 formed first mount 162 and slot 168 formed in the second mount 166 and is flexed rearward to form the aforementioned two strap loops or straps 160. The single strap body 161 may be fixedly secured within the slots 164 and 168 of the first and second mounts 162 and 166, respectively, or may be slidably mounted there through as discussed in more detail hereinbelow. As mentioned heretofore, alternative constructions and connections are, however, contemplated; for instance, the protective eyewear 100 may include one or more separate straps or other head securing structures tied to the strap mounts, stitched to the strap mounts, or attached to the strap mounts by a connector (e.g., a hook), for instance and without limitation.

The main body 110 of the protective eyewear 100 may be constructed from a variety of materials including plastics or polymers, metallic materials, etc. Similarly, the first and second eye portions 114 and 116 can be formed from a variety of transparent or non-transparent materials such as, but not limited to, shatterproof glass, clear plastics or polymers, etc. The main body 110 and the first and second eye portions 114 and 116, respectively, along with the strap 160 may be formed from hypo-allergenic materials to avoid harm to sensitive patients. Additionally, the main body 110, the first and second eye portions 114 and 116, respectively, and the head securing structure 158 may be formed from materials that are sterilizable either by hand or in a machine such as, but not limited to, an autoclave, to facilitate reuse of the protective eyewear 100. In some embodiments, the protective eyewear 100, or parts thereof, may be disposable. In some embodiments, the main body 110 may be formed into a single-piece unit, such as by plastics injection molding. The head securing structure 158 (e.g., the strap 160) may also be formed from a variety of materials including organic materials such as, for example cotton or wool or synthetic materials. Preferably, the synthetic materials are hypo-allergenic and sterilizable.

As best shown in FIG. 2, the fastener 130 of the present embodiment defines a mask-receiving space 132 for releasable engagement with the medical face mask 300. The fastener 130 can be configured to engage the medical face mask 300 in a variety of ways such as, but not limited to, a snap fit, a friction fit, etc., as discussed in more detail hereinbelow. While the illustrated fastener 130 is disclosed as being arcuate and configured to extend along the nosepiece 120, other shapes, both symmetrical and asymmetrical, such as, but not limited to, circular, rectangular, etc. are contemplated without departing from the scope of the present disclosure. Furthermore, while the fastener 130 is shown having an elongated configuration which extends substantially along the full length or perimeter of the nosepiece 120, and the mask-receiving space 132 is an elongate slot extending substantially along the full length or perimeter of the nosepiece 120, alternative embodiments are contemplated in which the fastener and/or mask-receiving space may be divided into, or include, two or more fasteners and/or mask-receiving spaces arranged at discrete positions along the length or perimeter of the nosepiece 120. In addition, while the fastener 130 is shown as being non-disconnectably attached to or integrally formed with the nosepiece 120, and the nosepiece 120 non-disconnectably attached to or integrally formed with adjacent portions (e.g., the central connecting portion 122) of the main body 110 of the protective eyewear 100, alternative embodiments are contemplated in which the fastener 130 may be disconnectably attached to the nosepiece 120, and/or the nosepiece 120 may be disconnectably attached to adjacent portions (e.g., the central connecting portion 122) of the main body 110 of the protective eyewear 100. For example, in one embodiment, the nosepiece 120 may include a clip configured to disconnectably attach the nosepiece 120 to the central connecting portion 122, and may further include the fastener 130 to disconnectably secure the nosepiece 120 to the medical face mask 300. In some embodiments, the disconnectable nosepiece 120 may be interchangeably attached to different face masks 300.

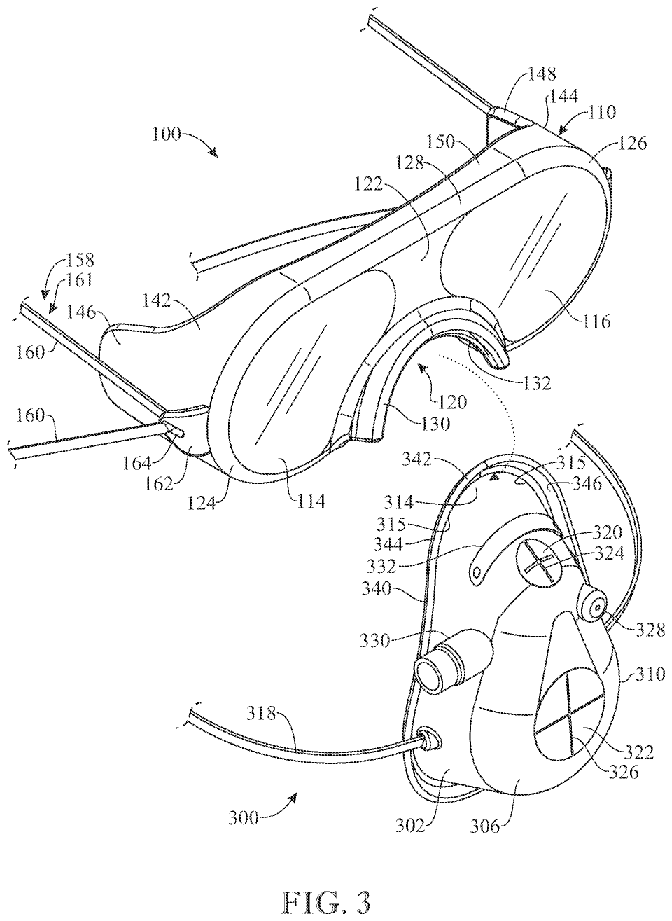

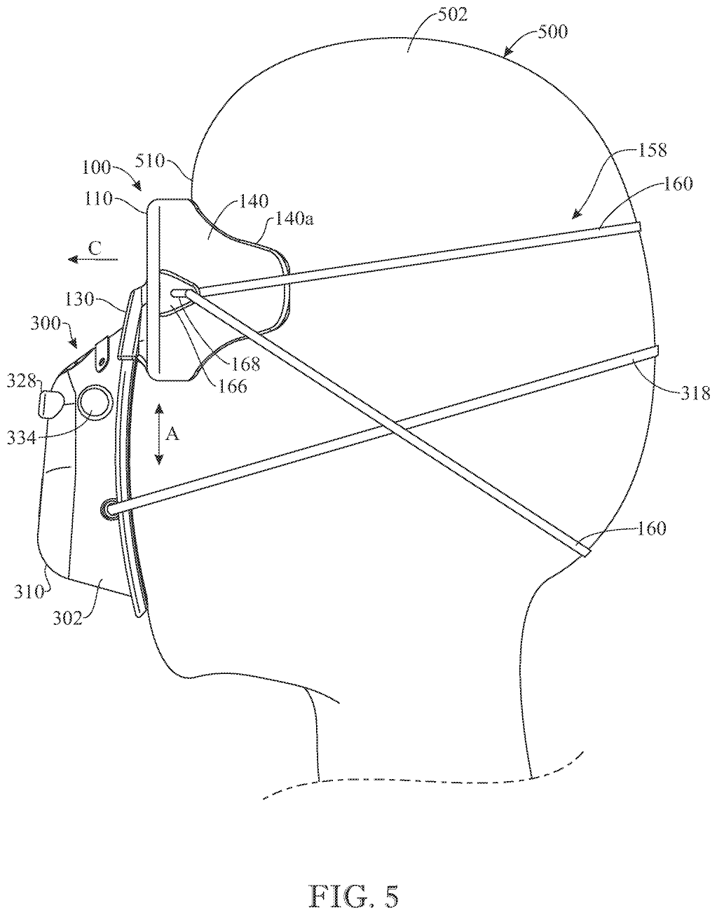

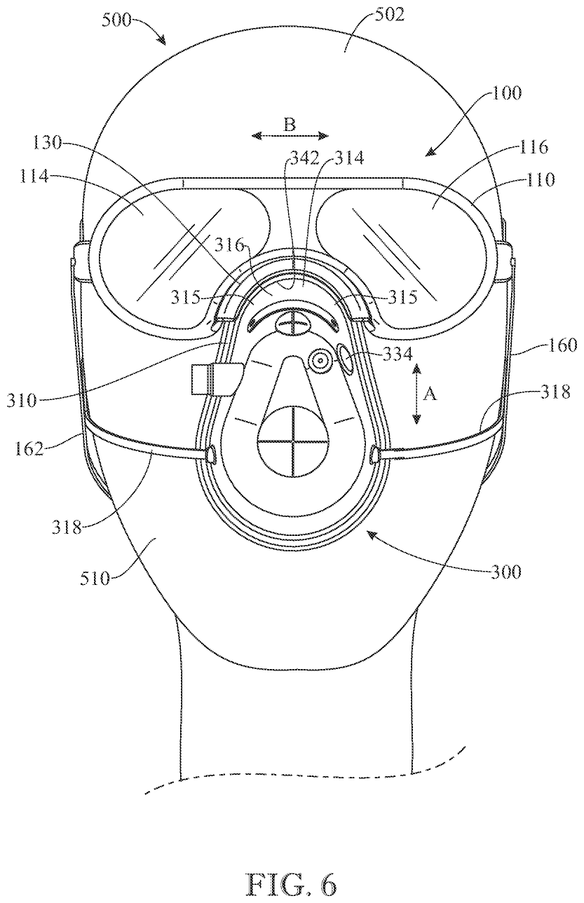

Referring for the movement to FIGS. 1 and 3, the medical face mask 300, in turn, is provided to supply anesthesia, oxygen, hydration, suction, etc. during a medical or surgical procedure, and to allow for the insertion of probes, tubes or other devices. It must be noted that, while a specific example of medical face mask 300 is shown herein, this example should not be construed as limiting; instead, alternative face masks or face mask variations, whether proprietary, third party, or standard, may be used without departing from the scope of the present disclosure. The medical face mask 300 depicted herein generally includes an elongated, cup-like mask body 310 configured to fit over the nose and the mouth and define an internal space or cavity 312 between the mask body 310 and the area of the face which is covered by the mask body 310. The medical face mask 300 further includes a strap 318 for securing the medical face mask 300 to the head of a patient. The medical face mask 300 may further include various ports, sections and/or implements carried by the mask body 310. Specifically, a nasal port 320 and an oral port 322 may be provided in a top central region and a lower central region of the mask body 310. The nasal and oral ports 320 and 322 may be formed, for instance, by a respective continuous, relatively thin membrane that may be perforatable, perforated, scored, tearable, or include a permanent opening, a tearable area, etc., all of which may facilitate accommodating nasotracheal or orotracheal intubations from outside the medical face mask 300 into the wearer's nose or mouth, respectively. For instance, in the depicted example, the nasal and oral ports 320 and 322 include a respective cross-shaped scored area 324 and 326, which can be easily torn to form a respective opening. The medical face mask 300 may include additional ports and/or connectors such as, but not limited to, a carbon dioxide sampling port 328, and a breathing therapy device connector 330 for allowing attachment a breathing facilitating device such as, but not limited to, an oxygen supply, a non-rebreather bag, a bubble humidifier, a humidifier, or a drug nebulizer. A malleable nose clip 332 may be provided on the mask body 310 to assist in retaining the mask body 310 about the nose of the patient. One or more exhaust ports 334, better shown in FIGS. 5 and 6, may be further provided on the mask body 310, to allow expired gases to be discharged from the internal cavity 312.

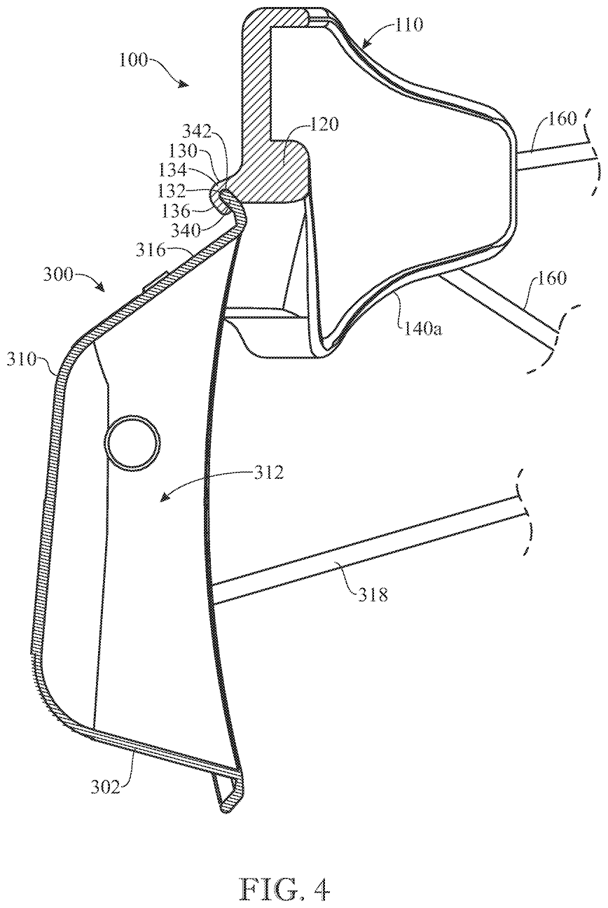

As best shown in FIGS. 3 and 4, the mask body 310 of the medical face mask 300 further includes a projecting ridge 340, configured to extend generally outwards from the wearer's face when wearing the medical face mask 300. In different embodiments, the ridge 340 may be provided along all or part of the perimeter of the mask body 310. For instance, the ridge 340 of the present medical face mask 300 extends along an entire periphery of the mask body 310, including along a top end 314 of the mask body 310, as best shown in FIG. 3. The ridge 340 may extend from an adjacent wall end portion 316 of the mask body 310, such that the wall end portion 316 and the ridge 340 extending therefrom form an angled, V-shaped, U-shaped, C-shaped, or other similar configuration in which the end portion 316 extends rearward (i.e. towards the wearer's face) and the ridge 340 extends frontward (i.e. away from the wearer's face) from the wall end portion 316.

With continued reference to FIGS. 3 and 4, the fastener 130 of the protective eyewear 100 is shaped and sized to engage with the ridge 340 of the medical face mask 300, such that engagement of the fastener 130 of the protective eyewear 100 with the ridge 340 of the medical face mask 300 releasably affixes the protective eyewear 100 to the medical face mask 300. In some embodiments, the fastener 130 may secure over an arcuate top portion 342 of the ridge 340. More specifically, the arcuate top portion 342 of the ridge 340 of the medical face mask 300 may fit into the mask-receiving space 132 defined in the arcuate engagement clip 130 of the protective eyewear 100 to releasably secure the protective eyewear 100 to the medical face mask 300. In some embodiments, the fastener 130 may have a U-shaped, C-shaped, V-shaped or other similar cross-section defining the mask-receiving space 132. As best shown in FIG. 4, the fastener 130 may include a forward and optionally downward extending, first portion 134, and a rearward and downward extending, second portion 136, which may extend from the first portion 134, the first and second portions 134 and 136 forming a U-shape, C-shape, V-shape or other configuration defining the mask-receiving space 132 therewithin. The first portion 134 may extend over the ridge 340 and contribute to prevent an upward migration of the mask body 310. The second portion 136 may extend below the ridge 340 and may contribute to reduce, or prevent, a downward migration of the mask body 310. Furthermore, as best shown in FIGS. 1 and 3, the engagement between the nosepiece 120 and the mask body 310 may extend to top side sections 315 of the mask body 310, to reduce, or prevent, a transverse migration of the mask body 310 along the wearer's face. In some embodiments, the engagement between the fastener 130 and the ridge 340 may be rigid, and completed such as by friction fitting, a hook-type fitting, or the like. In other embodiments, the engagement between the fastener 130 and the ridge 340 may be elastic, and may include a snap fitting, an elastic clip attachment, or the like.

Referring now to FIGS. 3-6, the use of the disclosed protective eyewear 100 to stabilize the medical face mask 300 on a patient 500 (FIGS. 5 and 6) will now be described. Initially, the eyewear 100 and the medical face mask 300 can be positioned on the head 502 of a patient separately or preassembled. As best shown in FIGS. 3 and 4, to assemble the eyewear 100 to the medical face mask 300, the ridge 340, and more specifically the arcuate top portion 342 of the ridge 340, is inserted into the mask-receiving space 132 of the fastener 130 provided on the nosepiece 120 of the eyewear 100. The arcuate top portion 342 of the ridge 340 is secured within the mask-receiving space 132 in elastic or non-elastic fashion, as mentioned heretofore. In this manner, the fastener 130 is snapped or affixed over the arcuate top portion 342 of the ridge 340, and preferably extending to top left and right sides 344 and 346 of the arcuate top portion 342 of the ridge 340, to secure the protective eyewear 100 to medical face mask 300 (FIG. 4).

Referring to FIG. 5, as noted above, the protective eyewear 100 and the medical face mask 300 may be positioned on the head 502 of the patient 500 separately or in a pre-assembled condition. The strap 318 of the medical face mask 300 is initially positioned about the head 502 of the patient 500 to secure the medical face mask 300 in place over a face 510 of the patient 500. Thereafter, the head securing structure 158 (e.g., straps 160) of the eyewear 100 are positioned over the head 502 of the patient 500 to both secure the eyewear 100 over the face 510 of the patient 500 and stabilize the medical face mask 300 on the face 510 of the patient 500.

As noted above, in the present embodiment, the single strap body 161 of the head securing structure 158 extends through the first and second mounts 162 and 166, respectively, provided on the main body 110 of the eyewear 100. The single strap body 161 may be affixed to the first and second mounts 162 and 166 or may be free to slide through the slots 164 and 168 defined by the first and second mounts 162 and 166, respectively. In this embodiment, as mentioned heretofore, the single strap body 161 forms an upper strap loop or strap 160 and a lower strap loop or strap 160 which surround the head 502 of the patient 500 to secure the protective eyewear 100 to the head 502 of the patient 500.

With continued reference to FIG. 5, in addition to the engagement of the fastener 130 of the eyewear 100 with the ridge 340 of the medical face mask 300, one or both straps 160 of the eyewear 100 may be secured over the strap 318 of the medical face mask 300, allowing to further stabilize the medical face mask 300 on the face 510 of the patient 500 and prevent migration of the medical face mask 300 about the face 510. For example, in the setup of FIG. 5, the lower strap 160 of the eyewear 100 is positioned over or outside of the strap 318 of the medical face mask 300, further preventing the medical face mask 300 from moving or migrating about the face 510 of the patient 500 during a procedure, while the upper strap 160 extends in a rearward and upward direction to further stabilize the protective eyewear 100.

As best shown in FIGS. 1, 5 and 6, the engagement of the fastener 130 of the eyewear 100 with the ridge 340 of the medical face mask 300 along with the overlapping securement of the head securing structure 158 (particularly, the lower strap 160) of the eyewear 100 over the strap 318 of the medical face mask 300 prevents the medical face mask 300 from migrating about the face 510 of the patient 300 in either the vertical direction indicated by arrow “A” (FIGS. 1, 5 and 6), the left-to-right or transverse direction indicated by arrow “B” (FIGS. 1 and 6), a forward direction indicated by arrow “C” (FIGS. 1 and 5), or in any combination thereof, when intubation, breathing devices and or other apparatus are attached to the different ports provided on the medical face mask 300 and such devices or apparatus exert pulling, pushing or torsion forces on the ports that may tend to displace the medical face mask 300.

In an alternative method of use of the protective eyewear 100 and face mask 300, the medical professional may not extend the strap 318 of the medical face mask 300 around the patient's head and instead only secure the protective eyewear 100 to the medical face mask 300 to reduce or prevent migration. In yet another implementation of the invention, the medical face mask 300 may not include a strap 318 and may be stabilized by the protective eyewear 100.

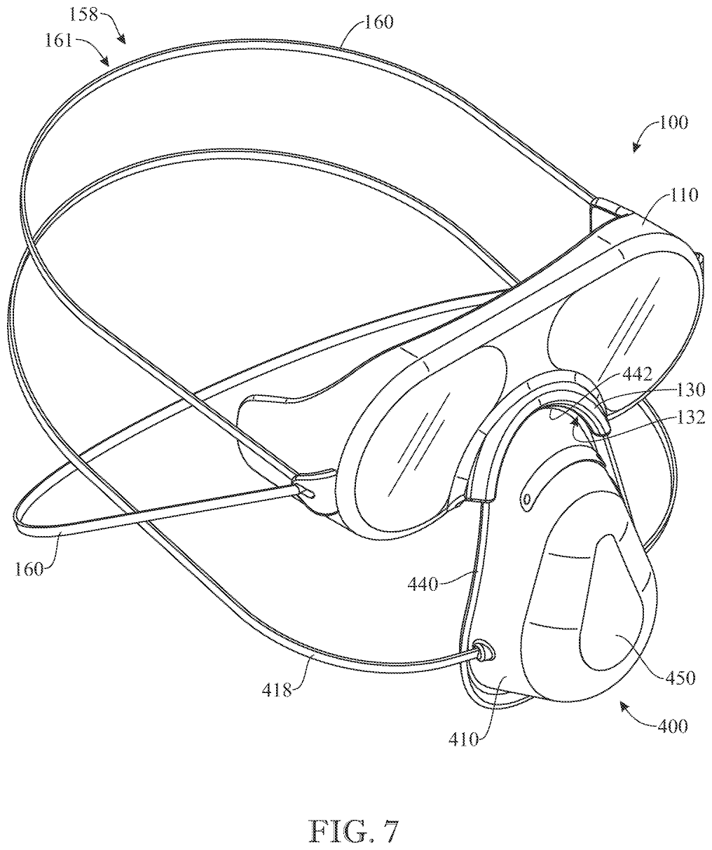

Turning now to FIG. 7, the disclosed protective eyewear 100 is illustrated affixed to an alternative medical face mask 400. As with medical face mask 300 above, the medical face mask 400 includes a mask body 410 having a ridge 440, and further includes a strap 418 attached to the mask body 410. The fastener 130 is releasably attachable to the ridge 440, and more specifically an arcuate top portion 442 of the ridge 440 is releasably received in the mask-receiving space 132 defined in the fastener 130 in a manner similar to that described hereinabove to secure the protective eyewear 100 to the medical face mask 400. The mask body 410 includes a perforatable membrane 450 over the mouth and nose of the patient to facilitate breathing. While not specifically shown, the lower strap loop or strap 160 of the eyewear 100 may be positioned over the strap 418 of the medical face mask 400 when placed on the head 502 (FIG. 5) of a patient 500 to stabilize the medical face mask 400 about the face 510 of the patient 500.

Alternative embodiments are contemplated without departing from the scope of the present disclosure. For example, the construction, parts, portions, sections, shape, size, proportions, or materials of the protective eyewear may vary.

Since many modifications, variations, and changes in detail can be made to the described preferred embodiments of the invention, it is intended that all matters in the foregoing description and shown in the accompanying drawings be interpreted as illustrative and not in a limiting sense. Thus, the scope of the invention should be determined by the appended claims and their legal equivalents.

What is claimed is:

1. A protective eyewear for stabilizing a medical face mask on a face of a subject, the protective eyewear comprising:
a main body configured to fit on a subject's face over at least one eye of the subject;
a head securing structure carried by the main body, the head securing structure configured to secure the main body to the subject's head; and
a fastener carried by the main body, the fastener defining one or more mask-receiving spaces for releasably receiving and securing of a portion of a medical face mask.

2. The protective eyewear of claim 1, wherein the main body comprises at least one eye portion configured to extend over and cover one or both eyes of the subject.

3. The protective eyewear of claim 2, wherein the main body is configured to enclose both eyes of the subject.

4. The protective eyewear of claim 1, wherein the head securing structure is elastic.

5. The protective eyewear of claim 1, wherein the head securing structure is configured to wrap around the subject's head.

6. The protective eyewear of claim 5, wherein the head securing structure comprises an upper elongated element and a lower elongated element defining a respective upper loop and lower loop extending from the main body and configured to wrap around the subject's head.

7. The protective eyewear of claim 1, wherein the main body comprises a nosepiece configured to extend over the subject's nose, and further wherein the fastener is provided at the nosepiece.

8. The protective eyewear of claim 7, wherein the one or more mask-receiving spaces comprise an elongated slot arcuately formed along the nosepiece.

9. The protective eyewear of claim 1, wherein the one or more mask-receiving spaces are configured to releasably receive a ridge of the medical face mask.

10. The protective eyewear of claim 1, wherein the one or more mask-receiving spaces are defined by a first portion and a second portion of the fastener, the first portion extending frontward from the main body, the second portion extending downward and rearward from the first portion and at an angle with the first portion.

11. The protective eyewear of claim 10, wherein the first portion extends downward from the main body.

12. A system for protecting the eyes of a subject and providing a stabilized medical face mask on the face of the subject, the system comprising:
a medical face mask comprising a mask body configured to fit over the nose and mouth of a subject, the medical face mask further comprising a strap extending from the mask body and configured to secure the mask body to the subject's head; and
a protective eyewear comprising:
a main body configured to fit on the subject's face over at least one eye of the subject;
a head securing structure carried by the main body, the head securing structure configured to secure the main body to the subject's head; and
a fastener carried by the main body, the fastener configured to attach to the mask body of the medical face mask.

13. The protective eyewear of claim 12, wherein the main body of the protective eyewear comprises a nosepiece configured to extend over the subject's nose, and further wherein the fastener is provided at the nosepiece and configured to releasably receive and secure a top portion of the mask body of the medical face mask.

14. The protective eyewear of claim 13, wherein the fastener comprises an elongated slot arcuately formed along the nosepiece, the elongated slot configured to releasably receive and secure an arcuate top portion of the mask body of the medical face mask.

15. A method of stabilizing a medical face mask on a face of a subject, comprising the steps of:
positioning a medical face mask on a subject's head, such that a mask body of the medical face mask is positioned over the nose and mouth of the subject;
positioning a protective eyewear on the subject's head, such that a main body of the protective eyewear covers at least one eye of the subject and a head securing structure of the protective eyewear secures the main body to the subject's head; and
preventing or reducing migration of the mask body of the medical face mask along the face of the subject by securing the protective eyewear to the mask body.

16. The method of claim 15, wherein the step of preventing or reducing migration of the mask body comprises securing the main body of the protective eyewear to the mask body of the medical face mask.

17. The method of claim 16, wherein the protective eyewear comprises a fastener configured to releasably attach to the mask body of the medical face mask, and wherein the step of preventing or reducing migration of the mask body comprises securing the fastener to the mask body.

18. The method of claim 17, wherein the fastener comprises one or more mask-receiving spaces, and further wherein the step of preventing or reducing migration of the mask body comprises releasably receiving and securing a portion of the mask body within the one or more mask-receiving spaces.

19. The method of claim 15, wherein the step of positioning the protective eyewear comprises securing a head securing structure of the protective eyewear to the subject's head, and further wherein the step of preventing or reducing migration of the mask body comprises overlapping the head securing structure of the protective eyewear over a strap of the medical face mask.

20. The method of claim 19, wherein the step of positioning the medical face mask comprises securing the strap of the medical face mask to the head of the subject.
